(19) United States
(12) Patent Application Publication
(10) Pub. No.: US 2023/0098378 A1
(43) Pub. Date: Mar. 30, 2023

(54) ARTICULATING ULTRASONIC SURGICAL INSTRUMENTS AND SYSTEMS

(71) Applicant: Covidien LP, Mansfield, MA (US)

(72) Inventors: James R. Fagan, Erie, CO (US); Thomas E. Drochner, Longmont, CO (US); Michael B. Lyons, Boulder, CO (US); David J. Van Tol, Boulder, CO (US); Matthew S. Cowley, Frederick, CO (US)

(21) Appl. No.: 17/911,363

(22) PCT Filed: Mar. 2, 2021
(86) PCT No.: PCT/US2021/020462

§ 371 Date: Sep. 13, 2022

Related U.S. Application Data
(60) Provisional application No. 63/004,141, filed on Apr. 2, 2020.

(51) Int. Cl. A61B 17/32

(52) U.S. Cl. CPC A61B 17/320092; A61B 2017/320094; A61B 2017/320074; A61B 2017/2947

(57) ABSTRACT

An articulating surgical end effector includes an expandable pivot defined at a distal end of an ultrasonic waveguide, a socket at least partially receiving the expandable pivot within an interior volume thereof, and a blade extending distally from the socket. The socket is configured to articulate about the expandable pivot in any direction, and the expandable pivot and socket together are configured to enable ultrasonic energy transmission through the ultrasonic waveguide to the blade in any articulated position of the socket relative to the expandable pivot.

### CROSS-REFERENCE TO RELATED APPLICATIONS

This application is a 371 National Stage Application of International Application No. PCT/US2021/020462, filed Mar. 2, 2021, which claims the benefit of U.S. Provisional Patent Application No. 63/004,141, filed Apr. 2, 2020, the entire contents of each of which are hereby incorporated herein by reference.

### BACKGROUND

### Technical Field

The present disclosure relates to surgical instruments and systems and, more particularly, to articulating ultrasonic surgical instruments and systems.

### Background of Related Art

Ultrasonic surgical instruments and systems utilize ultrasonic energy, i.e., ultrasonic vibrations, to treat tissue. More specifically, a typical ultrasonic surgical instrument or system includes a transducer configured to produce and transmit mechanical vibration energy at ultrasonic frequencies along a waveguide to an end effector configured to treat tissue, e.g., coagulate, cauterize, fuse, seal, cut, desiccate, fulgurate, or otherwise treat tissue. Traditionally, the transducer remains external of the surgical site, while the waveguide extends from the transducer into the surgical site to provide the ultrasonic energy to the ultrasonic end effector. The end effector is manipulated into position to treat a desired tissue or tissues.

Some ultrasonic surgical instruments and systems incorporate rotation features, thus enabling rotation of the ultrasonic end effector to a desired orientation within the surgical site. However, even in such instruments and systems, the ability to navigate within the surgical site via rotation and manipulation alone is limited.

### SUMMARY

As used herein, the term “distal” refers to the portion that is being described which is farther from a user, while the term “proximal” refers to the portion that is being described which is closer to a user. Further, to the extent consistent, any or all of the aspects of a particular device detailed herein may be used in conjunction with any or all of the other devices detailed herein.

One aspect of the disclosure is directed to an articulating ultrasonic surgical end effector including an expandable pivot defined at a distal end of an ultrasonic waveguide and a socket at least partially receiving the expandable pivot within an interior volume thereof. The socket is configured for articulation about the expandable pivot in any direction.

In another aspect of the disclosure, an ultrasonic surgical instrument includes a handle assembly, an ultrasonic transducer disposed within the handle assembly, an elongated body extending distally from the handle assembly, a waveguide extending at least partially through the elongated body, and an end effector disposed on a distal end of the waveguide. The end effector further includes an expandable pivot defined at a distal end of an ultrasonic waveguide and a socket at least partially receiving the expandable pivot within an interior volume thereof. The socket is configured for articulation about the expandable pivot in any direction.

In yet another aspect of the disclosure, the expandable pivot is configured to expand radially when ultrasonically excited.

In yet another aspect of the disclosure, the radial expansion of the expandable pivot transfers ultrasonic energy through the socket to the blade at any angle of articulation.

In yet another aspect of the disclosure, the expandable pivot is spherical in shape.

In yet another aspect of the disclosure, the socket is at least partially spherical in shape.

In yet another aspect of the disclosure, the blade extends from a centermost point of the at least partially spherical socket.

A surgical instrument provided in accordance with aspects of the present disclosure includes a handle assembly having an elongated body portion extending distally therefrom, and an articulating ultrasonic surgical end effector according to any of the above aspects, wherein the end effector extends distally from the elongated body portion.

Another aspect of the disclosure is directed to an articulating ultrasonic surgical end effector including a socket having an interior volume defined at a distal end of an ultrasonic waveguide, an expandable pivot configured to be at least partially inserted into the interior volume of the socket and for articulation within the socket in at least one direction, and a blade extending distally from the expandable pivot, such that the expandable pivot and socket are configured to enable ultrasonic energy transmission through the ultrasonic waveguide to the blade in an articulated position of the expandable pivot relative to the socket.

In yet another aspect of the disclosure, the expandable pivot is configured to expand radially when ultrasonically excited.

In yet another aspect of the disclosure, the radial expansion of the expandable pivot transfers ultrasonic energy from the expandable pivot to the socket at any articulated position.

In yet another aspect of the disclosure, the expandable pivot is spherical or cylindrical in shape.

In yet another aspect of the disclosure, the mating feature of the socket is at least partially spherical or cylindrical in shape.

In yet another aspect of the disclosure, the blade extends out from the expandable pivot along the central axis.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1A is a side, perspective view of an endoscopic surgical instrument configured for use in accordance with the aspects and features of present disclosure;

FIG. 1B is a side, perspective view of the endoscopic surgical instrument of FIG. 1A with the elongated body portion and jaw removed to illustrate internal components thereof;

FIG. 2 is a schematic illustration of a robotic surgical system configured for use in accordance with the aspects and features of present disclosure;

FIG. 3A is an enlarged, cross-sectional view of an articulating end effector, with the expandable pivot in an contracted state, in accordance with the present disclosure and configured for use with the endoscopic surgical instrument of FIG. 1A, the robotic surgical system of FIG. 2, or any other suitable surgical instrument or system;

FIG. 3B is an enlarged, side, perspective view of an articulating end effector, with the expandable pivot in an expanded state, in accordance with the present disclosure and configured for use with the endoscopic surgical instrument of FIG. 1A, the robotic surgical system of FIG. 2, or any other suitable surgical instrument or system;

FIG. 4A is a rear, perspective view of a socket having an open interior volume and an open proximal face;

FIG. 4B is a plan view of the socket having an open proximal face with diameter D_{S};

FIG. 4C is a plan view of an expandable pivot in an expanded state having diameter D_{P2};

FIG. 4D is a plan view of the expandable pivot in an contracted state having diameter D_{P1};

FIG. 5A is an enlarged, top view of the articulating end effector of FIG. 3A, with the ultrasonic blade and socket articulated to the left relative to the expandable pivot;

FIG. 5B is an enlarged, top view of the articulating end effector of FIG. 3A, with the ultrasonic blade and socket aligned or un-articulated relative to the expandable pivot;

FIG. 5C is an enlarged, top view of the articulating end effector of FIG. 3A, with the ultrasonic blade and socket articulated to the right relative to the expandable pivot;

FIG. 5D is an enlarged, top view of the articulating end effector of FIG. 3A, with the ultrasonic blade and socket articulated upward relative to the expandable pivot; and

FIG. 6 is an enlarged, side, perspective view of an articulating end effector, with a cylindrical expandable pivot and a curved planar socket, in accordance with the present disclosure and configured for use with the endoscopic surgical instrument of FIG. 1A, the robotic surgical system of FIG. 2, or any other suitable surgical instrument or system.

### DETAILED DESCRIPTION

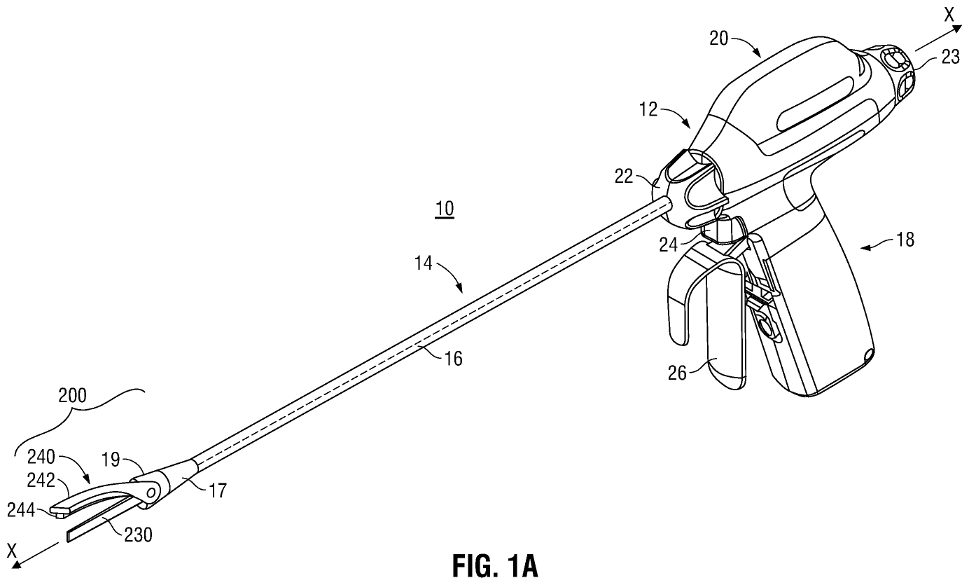

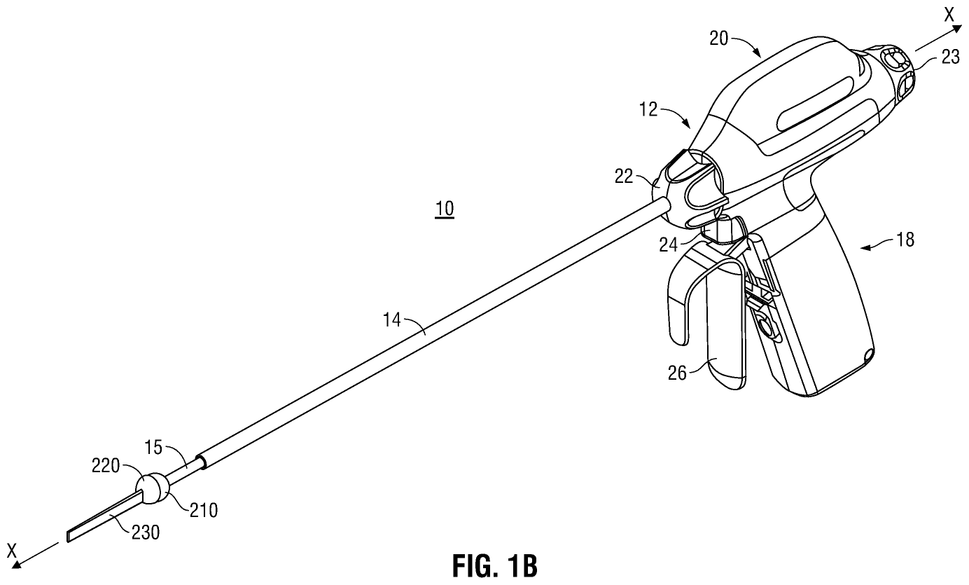

Referring to FIGS. 1A and 1B, an ultrasonic surgical instrument exemplifying the aspects and features of the present disclosure is shown generally identified by reference numeral 10. Although detailed with respect to ultrasonic surgical instrument 10, the aspects and features of the present disclosure are equally applicable for use with any suitable ultrasonic surgical instrument. For the purposes herein, ultrasonic surgical instrument 10 is generally described. Aspects and features of ultrasonic surgical instrument 10 not germane to the understanding of the present disclosure are omitted to avoid obscuring the aspects and features of the present disclosure in unnecessary detail.

Ultrasonic surgical instrument 10 generally includes a handle assembly 12, an elongated body portion 14 (FIG. 1A), a waveguide 15 (FIG. 1B), and an articulating surgical end effector 200.

Handle assembly 12 supports a battery assembly 18 and a transducer and generator assembly (“TAG”) 20 including an ultrasonic transducer and a surgical generator. Handle assembly 12 further includes a first rotation knob 22, a second rotation knob 23, an activation button 24, and a clamp trigger 26. Clamp trigger 26 of ultrasonic surgical instrument 10 is selectively manipulatable to actuate ultrasonic instrument 10. First rotation knob 22 is selectively manipulatable to rotate elongated body portion 14 and, thus, end effector 200 relative to handle assembly 12. Second rotation knob 23 is selectively manipulatable to actuate a motor, other powered drive mechanism, or a manual drive mechanism, e.g., gears, pulleys, tension cables, etc., to articulate end effector 200 relative to handle assembly 12. As an alternative to or in addition to first and second rotation knobs 22, 23, other suitable actuation mechanisms, e.g., toggle switches, joysticks, buttons, etc., may be provided to enable rotation and/or articulation.

Battery assembly 18 and TAG 20 cooperate, upon activation of activation button 24, to enable generation and transmission of ultrasonic energy for treating tissue therewith, e.g., to coagulate, cauterize, fuse, seal, cut, desiccate, fulgurate, or otherwise treat tissue, as detailed below. Battery assembly 18 and TAG 20 are each releasably secured to handle assembly 12, and are removable therefrom to facilitate disposal of handle assembly 12, with the exception of battery assembly 18 and TAG 20. However, it is contemplated that any or all of the components of ultrasonic surgical instrument 10 may be configured as disposable single-use components or sterilizable multi-use components, and/or that endoscopic surgical instrument 10 be connectable to a remote power source or generator rather than having such components on-board.

Elongated body portion 14 of ultrasonic surgical instrument 10 defines a longitudinal axis “X-X” (waveguide 15 may likewise be centered on longitudinal axis “X-X”) and includes a proximal shaft 16, an articulation section 17, and a distal support 19. Articulation section 17 extends between and interconnects proximal shaft 16 and distal support 19. In this manner, articulation of articulation section 17 relative to proximal shaft 16 articulates end effector 200 relative to proximal shaft 16 and handle assembly 12. Articulation section 17 may include one or more articulation components, e.g., articulation joint(s), articulation linkage(s), flexible portion(s), etc., coupled between proximal shaft 16 and distal support 19 to enable articulation of distal support 19 and, thus, end effector 200 relative to proximal shaft 16 in any radial direction, e.g., through 360 degrees about longitudinal axis “X-X” of proximal shaft 16. In other versions of the device, defined direction(s) of articulation, e.g., pitch and yaw, rather than infinite directions of articulation are also contemplated.

A jaw 240 is pivotably mounted on distal support 19 and a drive assembly (not shown) operably couples clamp trigger 26 of handle assembly 12 with jaw 240 of end effector 200 such that clamp trigger 26 is selectively actuatable to pivot jaw 240 relative to distal support 19 and blade 230 of end effector 200 between an open position and a clamping position for clamping tissue between jaw 240 and blade 230. The drive assembly may include a drive shaft, drive sleeve, drive cables, and/or other suitable components extending through handle assembly 12 and elongated body portion 14 to operably couple clamp trigger 26 with jaw 240 to enable pivoting of jaw 240 between the open and clamping positions regardless of the articulation of articulation section 17. Jaw 240 includes a more-rigid structural body 242 which is pivotably mounted on distal support 19, and a more-compliant jaw liner 244 secured to the more-rigid structural body 242 and positioned to oppose blade 230 to enable clamping of tissue therebetween.

The ultrasonic transducer of TAG 20 includes a plurality of piezoelectric elements or other suitable transducer component(s) configured to convert an electrical drive signal into ultrasonic vibration energy for transmission along waveguide 15 (see FIG. 1B) to blade 230. The generator of TAG 20, powered by battery 18 (or another power source), is configured to generate the electrical drive signal and provide the same to the ultrasonic transducer of TAG 20.

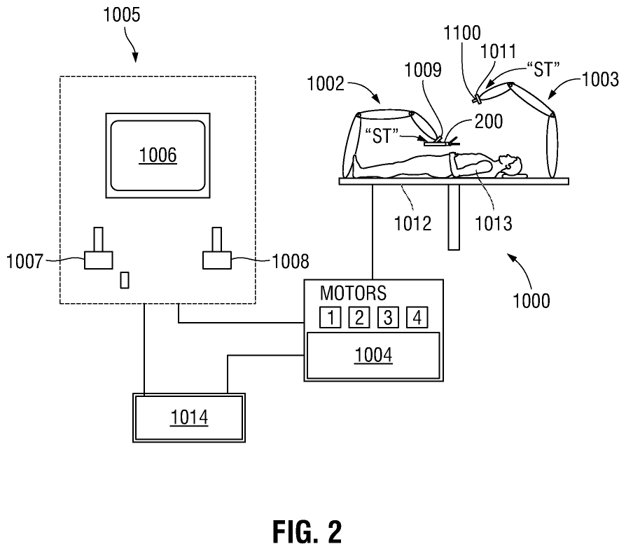

Referring to FIG. 2, an illustrative robotic surgical system exemplifying the aspects and features of the present disclosure is shown generally identified by reference numeral 1000. For the purposes herein, robotic surgical system 1000 is generally described. Aspects and features of robotic surgical system 1000 not germane to the understanding of the present disclosure are omitted to avoid obscuring the aspects and features of the present disclosure in unnecessary detail.

Robotic surgical system 1000 generally includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode. Robotic surgical system 1000 may be configured for use on a patient 1013 lying on a patient table 1012 to be treated in a minimally invasive manner. Robotic surgical system 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, a surgical tool “ST” supporting an end effector 200, 1100. End effector 200, as noted above with respect to endoscopic surgical instrument 10 (FIGS. 1A and 1B), and as described in greater detail below, is an articulating surgical end effector. End effector 1100 may be any other suitable surgical end effector, e.g., an endoscopic camera, other surgical tool, etc. Robot arms 1002, 1003 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and, thus, the surgical tools “ST” (including end effectors 200, 1100) execute a desired movement and/or function according to a corresponding input from manual input devices 1007, 1008, respectively. Control device 1004 may also be configured in such a way that it regulates the movement of robot arms 1002, 1003 and/or of the motors.

Referring again to FIGS. 1A and 1B, waveguide 15 includes a proximal end portion that is engaged with the ultrasonic transducer of TAG 20. Waveguide 15 extends from handle assembly 12 through proximal shaft 16 of elongated body portion 14 to articulation section 17. An expandable pivot 210 is defined at the distal end of waveguide 15. A socket 220 is operably coupled with expandable pivot 210 to enable articulation therebetween and includes ultrasonic blade 230 extending distally therefrom. Thus, ultrasonic blade 230 is articulatable relative to waveguide 15, e.g., via articulation of socket 220 relative to expandable pivot 210, while enabling transmission of ultrasonic energy from waveguide 15 to ultrasonic blade 230, e.g., through expandable pivot 210 and socket 220, at any articulated position. Expandable pivot 210 and socket 220 are at least partially disposed within articulation section 17 of elongated body portion 14. In this manner, articulation of articulation section 17 relative to proximal shaft 16 articulates socket 220 relative to expandable pivot 210, thereby articulating ultrasonic blade 230 relative to waveguide 15.

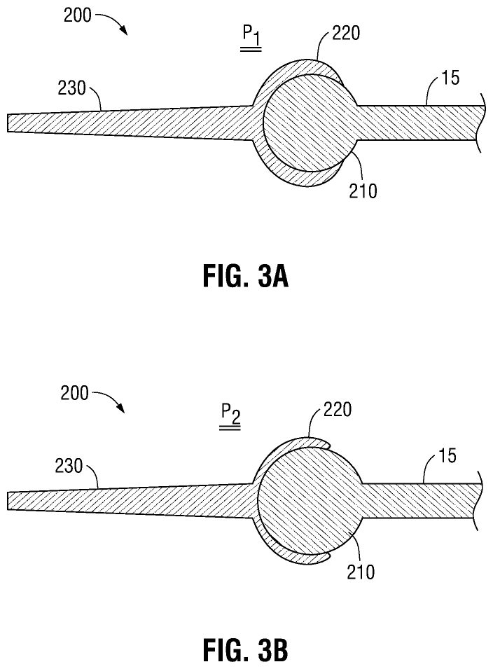

With additional reference to FIGS. 3A, and 3B, articulating surgical end effector 200, as noted above, includes a socket 220 formed at the proximal end of ultrasonic blade 230 of end effector 200. Socket 220 operably couples blade 230 of end effector 200 with waveguide 15. For example, socket 220 may couple to the distal end of waveguide 15 of endoscopic surgical instrument 10 (FIG. 1B) or the distal end of a waveguide (not explicitly shown) of attaching device 1009 of robot arm 1002 of robotic surgical system 1000 (FIG. 2), or to any other suitable surgical instrument or system.

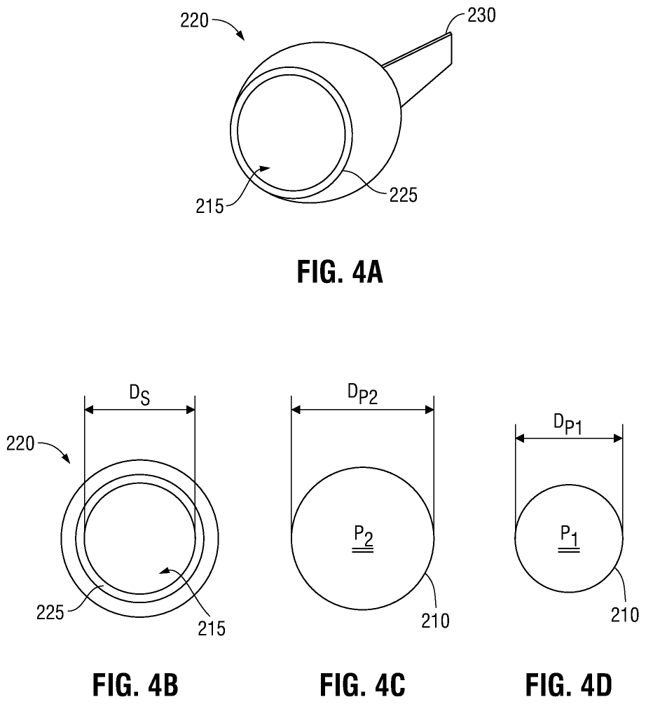

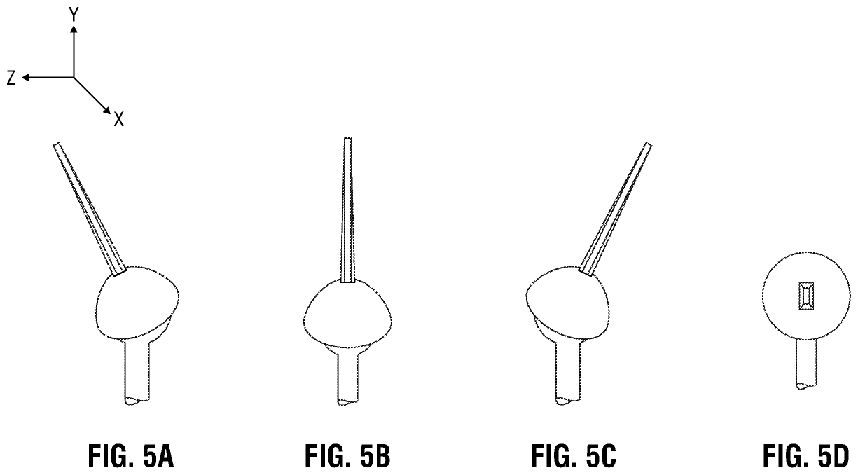

Articulating surgical end effector 200 includes expandable pivot 210, e.g., formed at a distal end of waveguide 15, at least partially enclosed within the socket 220. Socket 220 includes an interior volume 215 (FIG. 4A) and an open proximal face 225 (FIG. 4B). Blade 230 extends distally from socket 220 such that the blade 230 is oriented based on the articulated position of socket 220 about expandable pivot 210. In some aspects, both the expandable pivot 210 and the interior volume 215 of socket 220 are spherical in shape such that socket 220 can articulate about the outer surface of expandable pivot 210 unencumbered and in any direction. Furthermore, to maximize the range of motion about the pivot 210 in every direction, in some aspects the blade 230 extends from a centermost point (FIG. 5D) of the at least partially spherical socket 220. In other aspects, the relative positioning of the socket 220 and the expandable pivot 210 are reversed such that the socket 220 is defined at the distal end of waveguide 15 and the expandable pivot 210 is defined at the proximal end of the blade 230. In this reversed configuration, the blade 230 extends distally from expandable pivot 210, such that expandable pivot 210 can articulate while maintaining physical contact with inner surface of socket 220.

In use, when the ultrasonic transducer of TAG 20 transmits ultrasonic energy along waveguide 15, the expandable pivot 210 is stimulated to expand outward (radial expansion) and this expansion in turn transfers the ultrasonic energy to socket 220 and, ultimately, to the blade 230 at any angle of articulation. Accordingly, the expandable pivot 210 transitions between an contracted state P1 and an expanded state P2. More specifically, during ultrasonic energy transmission, expandable pivot 210 oscillates between the contracted state P1 and an expanded state P2. FIG. 3A shows the expandable pivot 210 in the contracted state P1, defining a smaller diameter, and, as such, the expandable pivot 210 does urge the interior volume 215 of socket 220 to expand and contract. This may be accomplished by designing the inner radius of the socket 220 to be substantially the same size as the expandable pivot 210 outer radius, so that there continuous contact between the socket 220 and expandable pivot 210.

In contrast, FIG. 3B shows where the expandable pivot 210 is in the expanded state P2, and as a result of the outward radial expansion of expandable pivot 210, the diameter thereof is increased to urge the interior volume 215 and, thus, socket 220 to expand radially outwardly, thereby transmitting the ultrasonic energy from the pivot 210 to the socket 220 for transmission to blade 230. The radial expansion of the expandable pivot 210 ensures that the ultrasonic energy is transferred to the socket 220 and blade 230 regardless of the articulated position of the blade 230.

Turning to FIGS. 4A-D, in aspects, the open proximal face 225 of socket 220 is circular, while pivot 210 and interior volume 215 of socket 220 are both spherical. The interior volume 215 of socket 220 is configured to at least partially enclose the expandable pivot 210 regardless of whether the expandable pivot 210 is in the contracted state P1 or the expanded state P2 and regardless of the articulated position thereof. To ensure that the expandable pivot 210 remains securely contained within the interior volume 215 of socket 220, the open proximal face 225 has a diameter D_{S} smaller than the diameter D_{P1} of the expandable pivot 210 in the contracted state P1; i.e., D_{S}<D_{P1}. The diameter D_{P2} of the expandable pivot 210 in the expanded state P2 is larger than the diameter D_{P1} of the expandable pivot 210 in the contracted state P1; i.e., D_{P1}<D_{P2}. Therefore, the diameter D_{S} of open proximal face 225 is also less than the diameter D_{P2} of the expandable pivot 210 in the expanded state P2; i.e., D_{S}<D_{P1}<D_{P2}. Furthermore, in some devices, socket 220 may retain the ability to articulate about the outer surface of expandable pivot 210, regardless of whether the expandable pivot 210 is in the contracted state P1, or in the expanded state P2. In other devices, expandable pivot 210, in the expanded state P2, may lock the articulated position of blade 230 relative to waveguide 15 (FIGS. 3A and 3B)

Referring now to FIGS. 5A-D, the range of motion of blade 230, via articulation of socket 22 about pivot 210, is shown to enable articulation in an unlimited number of directions.

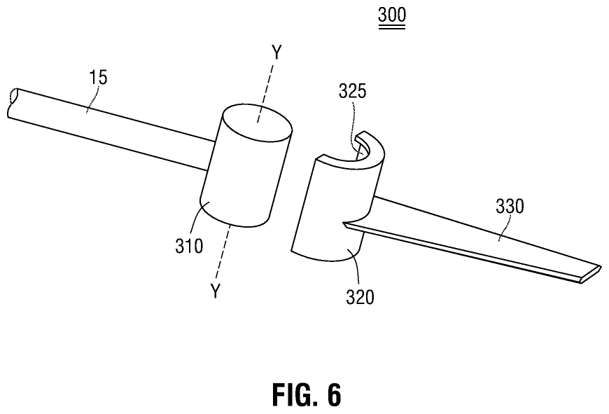

In other aspects of articulating surgical end effectors, the expandable pivot and the socket need not be spherical. FIG. 6 shows an articulating surgical end effector 300 including a substantially cylindrical expandable pivot 310 formed at a distal end of waveguide 15. Expandable pivot 310 is at least partially enclosed by a curved socket 320 formed at the proximal end of an ultrasonic blade 330, e.g., socket 320 defines a portion of an outer wall of a hollow cylinder. Socket 320 and expandable pivot 310 are operably coupled in continuous contact to facilitate articulation of socket 320 about an axis “Y-Y” defined through expandable pivot 310, although other orientations of articulation are also contemplated. Socket 320 includes an inner surface 325 shaped to follow the curvature of expandable pivot 310, such that socket 320 and expandable pivot 310 maintain physical contact with one another during articulation. Additionally, ultrasonic blade 330 extends distally from socket 320 such that the blade 330 is oriented based on the articulated position of socket 320 about expandable pivot 310. Further, in some aspects, the blade 330 extends from a center point of the socket 320.

Moreover, in still other aspects, the relative positioning of the socket 320 and the expandable pivot 310 can be reversed such that the socket 320 is defined at the distal end of waveguide 15 and the expandable pivot 310 is defined at the proximal end of the ultrasonic blade. In this reversed configuration, the blade 330 extends distally from expandable pivot 310, and expandable pivot 310 can articulate about axis “Y-Y” while maintaining physical contact with inner surface 325 of socket 320.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An articulating ultrasonic surgical end effector, comprising:
an expandable pivot defined at a distal end of an ultrasonic waveguide;
a socket at least partially receiving the expandable pivot within an interior volume thereof, the socket configured for articulation about the expandable pivot in any direction; and
a blade extending distally from the socket, where the expandable pivot and socket are configured to enable ultrasonic energy transmission through the ultrasonic waveguide to the blade in any articulated position of the socket relative to the expandable pivot.

2. The articulating ultrasonic surgical end effector according to claim 1, wherein the expandable pivot is configured to expand radially when ultrasonically excited.

3. The articulating ultrasonic surgical end effector according to claim 2, wherein the radial expansion of the expandable pivot transfers ultrasonic energy from the expandable to the socket at any articulated position.

4. The articulating ultrasonic surgical end effector according to claim 3, wherein the expandable pivot is spherical in shape.

5. The articulating ultrasonic surgical end effector according to claim 4, wherein the mating feature of the socket is at least partially spherical in shape.

6. The articulating ultrasonic surgical end effector according to claim 5, wherein the blade extends out from the socket along the central axis.

7. An ultrasonic surgical instrument, comprising:
a handle assembly;
an ultrasonic transducer disposed within the handle assembly;
an elongated body extending distally from the handle assembly;
a waveguide extending at least partially through the elongated body; and
an end effector disposed on a distal end of the waveguide, the end effector comprising:
an expandable pivot defined at a distal end of an ultrasonic waveguide;
a socket at least partially receiving the expandable pivot within an interior volume thereof, the socket configured for articulation about the expandable pivot in any direction; and
a blade extending distally from the socket, where the expandable pivot and socket are configured to enable ultrasonic energy transmission through the ultrasonic waveguide to the blade in any articulated position of the socket relative to the expandable pivot.

8. The ultrasonic surgical instrument according to claim 7, wherein the expandable pivot is configured to expand radially when ultrasonically excited.

9. The ultrasonic surgical instrument according to claim 8, wherein the radial expansion of the expandable pivot transfers ultrasonic energy through the socket to the blade at any angle of articulation.

10. The ultrasonic surgical instrument according to claim 9, wherein the expandable pivot is spherical in shape.

11. The ultrasonic surgical instrument according to claim 10, wherein the mating feature of the socket is at least partially spherical in shape.

12. The ultrasonic surgical instrument according to claim 11, wherein the blade extends from a centermost point of the at least partially spherical socket.

13. An articulating ultrasonic surgical end effector, comprising:
a socket defined at a distal end of an ultrasonic waveguide, the socket including an interior volume;
an expandable pivot configured to be at least partially inserted into the interior volume of the socket, the expandable pivot configured for articulation within the socket in at least one direction; and
a blade extending distally from the expandable pivot, where the expandable pivot and socket are configured to enable ultrasonic energy transmission through the ultrasonic waveguide to the blade in an articulated position of the expandable pivot relative to the socket.

14. The articulating ultrasonic surgical end effector according to claim 13, wherein the expandable pivot is configured to expand radially when ultrasonically excited.

15. The articulating ultrasonic surgical end effector according to claim 14, wherein the radial expansion of the expandable pivot transfers ultrasonic energy from the expandable pivot to the socket at any articulated position.

16. The articulating ultrasonic surgical end effector according to claim 15, wherein the expandable pivot is spherical or cylindrical in shape.

17. The articulating ultrasonic surgical end effector according to claim 16, wherein the mating feature of the socket is at least partially spherical or cylindrical in shape.

18. The articulating ultrasonic surgical end effector according to claim 17, wherein the blade extends out from the expandable pivot along the central axis.
